(19) United States
(12) Patent Application Publication
(10) Pub. No.: US 2007/0009557 A1
(43) Pub. Date: Jan. 11, 2007

(54) Moldable implant material

(75) Inventors: Klaus-Dieter Kuhn, Marburg (DE); Sebastian Vogt, Erfurt (DE)

Correspondence Address: Kurt G. Briscoe;Norris, McLaughlin & Marcus P.A., 18th Floor, 875 Third Avenue, New York, NY, 10022, US

(73) Assignee: Heraeus Kulzer GmbH, Hanau (DE)

(21) Appl. No.: 11/471,987

(22) Filed: Jun. 21, 2006

(30) Foreign Application Priority Data
Jun. 22, 2005 (DE) 10 2005 029 206.2

(51) Int. Cl. A61K 9/00

(52) U.S. Cl. 424/400

(57) ABSTRACT

A moldable implant material is described which is characterized in that a biodegradable or biocompatible monofilament or polyfilament thread is formed e.g. of polyester, polyamide, a corrodible iron alloy, magnesium, magnesium alloys, polysaccharides, polysaccharide derivatives, proteins, and/or protein derivatives or of combinations of these materials in such a way that, at a distance of 3-30 mm along the thread respectively, a circular loop and/or several circular loops are formed which have respectively a joint point of origin along the thread, at least 3 consecutive loops being present along the thread.

A moldable implant material is described which is intended for temporarily filling wound cavities, in particular bone defects.

Bone defects frequently occur in surgery after repairing bone cysts, after the extirpation of tumors and after surgical repair of bone infections. In an ideal case, the cavities formed ought to be closed up by newly formed bone building up in order to restore the natural state. However, bone tissue does not build up to close the bone cavity in the case of fairly large, so-called critical size defects. One possibility for treating bone defects is augmentation with autologous bone material. The quantity of autologous bone material is naturally limited for every patient and requires an additional second surgical intervention which is accompanied by risks. As an alternative to autologous bone material, synthetically available bone substitute materials can also be used instead of the autologous bone material. An important group is ceramic bone substitute materials which are used in the form of granules or in the form of so-called beads, among other things. Typical ceramic bone substitute materials are described in DE 196 14 421, DE 100 63 119, W09107357 and W02004112855. However, a problem in the case of granules is the fact that, at best, granules can be fixed vis-a-vis each other by enmeshing in the bone defect. In this way, granules can migrate in the bone defect and distribute unevenly in the bone defect. Bone regeneration may therefore be impaired. Fixing ceramic bone substitute materials by collagen non-wovens or gelatine non-wovens, too, is not particularly promising because they dissolve after a few days.

The invention is based on the object of providing an implant material which can be used to fill wound cavities, in particular bone defects. This implant material is to be such that, after introduction into bone cavities, it fills the space of the cavity as a result of its structure, without collapsing. The implant material is to be suitable for spatial fixing of ceramic bone substitute materials.

The object of the invention was achieved by developing a moldable implant material which is characterized in that a biodegradable or biocompatible monofilament or polyfilament fiber is formed in such a way that, at a distance of 2-30 mm respectively along the thread, a circular loop and/or several circular loops which have a joint point of origin along the thread are formed, at least 3 consecutive loops being present along the thread. A loop should be understood to be an approximately circular or elliptical formation of the thread.

Preferably, the implant consists of polyester, polyamide, a corrodible iron alloy, surgical steel, magnesium, magnesium alloys, polysaccharides, polysaccharide derivatives, proteins, protein derivatives or of combinations of these materials.

When using corrodible iron alloys, magnesium or magnesium alloys, the implant material is capable of plastic deformation. The implant material according to the invention is, surprisingly enough, capable of elastic deformation in the case of the use of polyesters and polyamides.

The subject matter of the invention also consists of non-textile flat materials in the form of knitted fabrics, felts and non-wovens containing loop structures and/or knots.

It is advantageous that the loops can be displaced along the thread.

Appropriately, the loops are knotted, if necessary.

Within the framework of the invention, it is also advantageous that one or several pharmaceutical active agents are applied on to the surface of the implant material. These active agents can be present in the form of wax-type active agents such as gentamicin palmitate or gentamicin stearate which adhere without using polymeric film formers. It corresponds also to the meaning of the invention that non-film forming, non-adhesive active agents are enclosed in the low molecular, well adhering active agents or auxiliary agents. Saturated fats and saturated fatty acids, in particular, can be considered as suitable low molecular auxiliary agents. The use of tripalmitin and tristearin as auxiliary agent is particularly preferred. Antibiotics, antiphlogistics, hormones and bisphosphonates can be considered for use as active agents.

It is appropriate that the loops enclose annular bodies which are arranged radially around the thread axis. These bodies may be present in the form of spheres or rollers which contain one or several bores.

It is also appropriate for the loops to be enclosed in spherical or roller-shaped bodies. The circular loops can be present in the form of an open circle. An open circle should be understood to have a form similar to that of the letter U. The spherical or roller shape bodies can be applied onto the loop by pressing in such a way that the bodies enclose the loops completely or partially. Surprisingly, the loops prevent slipping of the bodies along the axis of the thread effectively.

It is appropriate that the annular or spherical or roller-shaped bodies consist of 1-tricalcium phosphate, a-tricalcium phosphate, octacalcium phosphate, rhenanite, sodium potassium calcium phosphates, calcium sulphate dihydrate, calcium carbonate, zirconium dioxide or of combinations of these substances or combinations of these substances and organic substances from the group of polyesters, polyamides, polymethacrylates, polyacrylates, proteins and of saturated fats.

Moreover, it is appropriate that the annular or spherical or roller-shaped bodies contain at least one pharmaceutical active agent from the group of antiinfectives, antiphlogistics, cytostatics, bisphosphonates and growth factors.

The annular or spherical or roller-shaped bodied contain preferably contain antiinfectives from the group of aminoglycoside antibiotics, lincosamide antibiotics, fluoroquinolone antibiotics, streptogramin antibiotics, makrolide antibiotics, ketolide antibiotics, steroid antibiotics, oxazolidinone antibiotics and nitroimidazols.

It is appropriate that the annular or spherical or roller-shaped bodies release pharmaceutical active agents in the aqueous medium.

It is appropriate for at least 3 or several annular or spherical or roller-shaped bodies to be present. An implant material is particularly preferred in the case of which 30, 40 or 60 bodies are fixed along the thread axis.

The use of the moldable implant material takes place according to the invention by the moldable implant material being provided as a medicinal product or a pharmaceutical.

### BRIEF DESCRIPTION OF THE DRAWINGS

FIG. 1 shows a loop arrangement according to the invention with 7 loops at equal distances.

FIG. 2 shows an alternative loop arrangement according to the invention with 7 loops at equal distances.

FIG. 3 shows the loop arrangement of FIGS. 1 and 2 with additional annular bodies (shaded) which are enclosed by the loops and arranged radially around the thread axis.

FIG. 4 shows an arrangement in the case of which the loops are enclosed in essentially spherical or roller-shaped bodies (shaded).

The invention will now be explained by the following examples 1-5 without restricting the invention.

### EXAMPLE 1

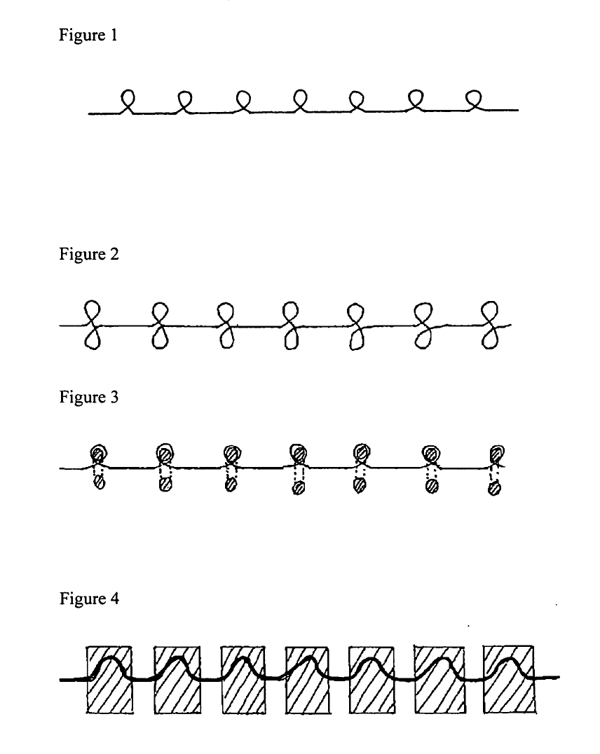

A metal plate with 10 pins (diameter 6 mm, distance of the pins 10 mm) which are arranged in a row, is used as carrier for the production of the implant materials. A PCL thread (polycaprolactone co-L-lactide thread, USP 2-0) is applied onto the plate and looped once around the pins respectively. The plate with the PCL threads fixed thereon is then heated in the drying cabinet to 70° C. and subsequently cooled to room temperature. Subsequently, the PCL thread is withdrawn from the pins. The PCL thread contains loops with a diameter of 5-6 mm at a distance of 10 mm respectively. After cooling, the loops are fixed at room temperature (diagrammatic representation in FIG. 1).

### EXAMPLE 2

A metal plate with 20 pins (diameter 6 mm, distance of the pins 10 mm), the pins being arranged in two opposite rows of 10 pins and the distance of the rows being 12 mm, is used as carrier for the production of the implant material. A PCL thread (polycaprolactone co-L-lactide thread, USP 2-0) is applied onto the plate and looped once around the pins respectively, the thread axis being between the two rows of pin. The plate with the PLC thread fixed thereon is then heated in the drying cabinet to 70° C. and subsequently cooled to room temperature and the PCL thread is withdrawn from the pins. The PCL thread exhibits two opposing loops at a distance of 10 mm respectively which originate respectively at a joint point along the thread axis. The loops have a diameter of 5-6 mm. After cooling, the loops are fixed at room temperature (diagrammatic representation in FIG. 2).

### EXAMPLE 3

A PCL thread (USP 2-0) with loops according to example 1 contains an annular body in each loop. The annular bodies have a mass of 220 mg, an outside diameter of 6 mm and a height of 5.8 mm. The bodies are composites of 17.31% by mass calcium carbonate, 69.23% by mass calcium sulphate dihydrate, 11.80% by mass tripalmitin and 1.66% by mass gentamicin sulphate AK 600 (1.0% by mass gentamicin base) (diagrammatic representation in FIG. 3).

### EXAMPLE 4

A PCL thread (USP 2-0) with loops according to example 2 contains an annular body in each loop. The annular bodies have a mass of 220 mg, an outside diameter of 6 mm and a height of 5.8 mm. The bodies are composites of 17.40% by mass calcium carbonate, 69.61% by mass calcium sulphate dihydrate, 11.88% by mass tripalmitin and 1.11% by mass clindamycin hydrochloride AK 896 (1.0% by mass clindamycin base).

### EXAMPLE 5

On a PCL thread (USP 2-0) with loops according to Example 1, an approximately spherical body, starting out from a powder, is pressed at room temperature by means of a modified tablet press onto each loop. The compression force is approximately 5 metric tons. The powder is composed of 17.31% by mass calcium carbonate, 69.23% by mass calcium sulphate dihydrate, 11.80% by mass tripalmitin and 1.66% by mass gentamicin sulphate AK 600 (1.0% by mass gentamicin base). The mass of the spherical bodies is 250 mg (diagrammatic representation in FIG. 4).

### EXAMPLE 6

On a PCL thread (USP 2-0), an approximately spherical body, starting out from a powder, is pressed at room temperature by means of a modified tablet press onto each loop. The compression force is approximately 5 metric tons. The thread is introduced into the matrix in such a way that the thread enters the powder to be pressed not in the centre but at approx. 60-70 percent of the level of fill. The powder is composed of 17.31% by mass calcium carbonate, 69.23% by mass calcium sulphate dihydrate, 11.80% by mass tripalmitin and 1.66% by mass gentamicin sulphate (1.0% by mass gentamicin base). On pressing, a loop of the PCL thread is formed in each individual molded body. The mass of the spherical body is 250 mg.

What is claimed is:

1. Moldable implant material in the form of a biodegradable or biocompatible, monofilament or polyfilament thread which exhibits, at a distance of 3-30 mm respectively, a circular loop and/or several circular loops which have a joint point of origin along the thread, at least 3 consecutive loops being present along the thread.

2. Moldable implant material according to claim 1 of polyester, polyamide, a corrodible iron alloy, surgical steel, magnesium, magnesium alloys, polysaccharides, polysaccharide derivatives, proteins, protein derivatives or of combinations of these materials.

3. Moldable implant material according to claim 1, wherein the loops are knotted.

4. Moldable implant material according to claim 1, wherein at least one pharmaceutical active agent is applied to its surface.

5. Moldable implant material according to claim 1, which additionally exhibits annular bodies which are enclosed by the loops and arranged radially around an axis of the thread.

6. Moldable implant material according to claim 1, wherein the loops are enclosed in essentially spherical or roller-shaped bodies.

7. Moldable implant material according to claim 6, wherein the annular or spherical or roller-shaped bodies consist of 1-tricalcium phosphate, a-tricalcium phosphate, octacalcium phosphate, rhenanite, sodium potassium calcium phosphates, calcium sulphate dihydrate, calcium carbonate, zirconium dioxide or of combinations of these substances or combinations of these substances and organic substances from the group of polymethacrylates, polyacrylates, polyesters, polyamides, proteins, and saturated fats.

8. Moldable implant material according to claim 7, wherein the annular or spherical or roller-shaped bodies comprise at least one pharmaceutical active agent from the group of antiinfectives, antiphlogistics, cytostatics, bisphosphonates and growth factors.

9. Moldable implant material according to claim 7, wherein the annular or spherical or roller-shaped bodies comprise antiinfectives from the group of aminoglycoside antibiotics, lincosamide antibiotics, fluoroquinolone antibiotics, streptogramin antibiotics, makrolide antibiotics, ketolide antibiotics, steroid antibiotics, oxazolidinone antibiotics and nitroimidazols.

10. Moldable implant material according to claim 7, wherein the annular or spherical or roller-shaped bodies release pharmaceutical active agents in the aqueous medium.

11. Moldable implant material according to claim 7, wherein at least three or several annular or spherical or roller-shaped bodies are present.

12. A medicinal or pharmaceutical product comprising a moldable implant material according to claim 1.

13. A medicinal or pharmaceutical product comprising a moldable implant material according to claim 7.

14. A method of temporarily filling a wound cavity comprising filling the wound cavity with a moldable implant material according to claim 1.

15. A method of temporarily filling a wound cavity comprising filling the wound cavity with a moldable implant material according to claim 7.
